(19) United States
(12) Patent Application Publication
(10) Pub. No.: US 2025/0090004 A1
(43) Pub. Date: Mar. 20, 2025

(54) SUCTION VALVE CARTRIDGE FOR AN ENDOSCOPE

(71) Applicant: Boston Scientific Scimed, Inc., Maple Grove, MN (US)

(72) Inventor: Juan M. Alvarez Azpeitia, Framingham, MA (US)

(73) Assignee: Boston Scientific Scimed, Inc., Maple Grove, MN (US)

(21) Appl. No.: 18/888,587

(22) Filed: Sep. 18, 2024

Related U.S. Application Data
(60) Provisional application No. 63/583,980, filed on Sep. 20, 2023.

(51) Int. Cl. A61B 1/015; A61B 1/00

(52) U.S. Cl. CPC A61B 1/015; A61B 1/00094; A61B 1/00137

(57) ABSTRACT

Devices, systems, and methods for a suction valve assembly for a medical device. The suction valve assembly includes a cylindrical valve body, a valve cap, a sliding shaft, and a lower seal. The valve body has a vent opening at the top, a working opening in at bottom, and a suction source opening in the side surface. The valve cap, sliding shaft, and lower seal move vertically relative to the valve body between an upper position, where the lower seal is seated against the valve body and obstructs the working opening, and a lower position, wherein the lower seal is below the working opening and allows suction through the suction source opening.

### CROSS-REFERENCE TO RELATED APPLICATIONS

This application claims the benefit of U.S. Provisional Patent Application Ser. No. 63/583,980 filed on Sep. 20, 2023, the disclosure of which is incorporated herein by reference.

### FIELD

This disclosure relates generally to valve assemblies and methods, and particularly to suction valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a suction valve assembly for a medical device includes a cylindrical valve body having a top, a bottom, and a side surface, the valve body having a vent opening in the top, a working opening in the bottom, and a suction source opening in the side surface; a valve cap; a sliding shaft attached to the valve cap, the sliding shaft and valve cap moving vertically relative to the valve body between an upper position and a lower position; and a lower seal attached to the sliding shaft, the seal seating against and obstructing the working opening when the sliding shaft is in the upper position, the lower seal below and not obstructing the working opening when the sliding shaft is in the lower position.

Alternatively or additionally to any of the examples above, the suction valve assembly can further include an upper seal moving with the sliding shaft and valve cap, the upper seal seating against and obstructing the vent opening when the sliding shaft is in the lower position, the upper seal above and not obstructing the vent opening when the sliding shaft is in the upper position.

Alternatively or additionally to any of the examples above, the upper seal can be attached to the valve cap.

Alternatively or additionally to any of the examples above, the upper seal can be attached to the sliding shaft.

Alternatively or additionally to any of the examples above, the valve body can have a sliding shaft channel housing the sliding shaft.

Alternatively or additionally to any of the examples above, the valve body can have a vertical air channel extending between the vent opening and the working opening and a side channel extending between the vertical air channel and the suction source opening.

Alternatively or additionally to any of the examples above, the sliding shaft channel can be separated from the vertical air channel such that the sliding shaft channel is not in fluid communication with the vent opening, working opening, or suction source opening.

Alternatively or additionally to any of the examples above, the sliding shaft channel can overlap the vertical air channel such that the sliding shaft channel is in fluid communication with the vent opening, working opening, or suction source opening.

Alternatively or additionally to any of the examples above, the suction valve assembly can also include a spring member positioned between the valve body and the valve cap, the spring member biased to push the valve cap and sliding shaft upward into the upper position.

Alternatively or additionally to any of the examples above, the suction valve assembly can also include a rigid insert positioned within the valve body, the rigid insert having a slide shaft opening for movement of the slide shaft therethrough and an air opening for movement of air therethrough when the slide shaft is in the upper position. The valve body can be made of a flexible material having a lower durometer than the rigid insert.

Alternatively or additionally to any of the examples above, the suction valve assembly can also include a first outer seal positioned on the side surface of the valve body above the suction source opening and a second outer seal positioned on the side surface of the valve body below the suction source opening.

Alternatively or additionally to any of the examples above, at least one of the first and second outer seals can be a radial o-ring.

As another example, an endoscopic surgical device can include an endoscopic probe having a working channel; a suction valve assembly according to any of the examples above where the working opening of the valve body is in fluid communication with the working channel of the endoscopic probe; and a source of suction in fluid communication with the suction source opening of the suction valve assembly, such that, when the sliding shaft of the valve is in the lower position, it provides suction to the working channel of the endoscopic probe from the suction source opening, through the valve body, and into the working channel opening.

Alternatively or additionally to any of the examples above, the endoscopic surgal device can also include an endoscopic handle having a suction valve location with an inner wall, the side surface of the body valve of the suction valve assembly pressing tightly against the inner wall of the suction valve location of the endoscopic handle.

Alternatively or additionally to any of the examples above, when the sliding shaft of the valve is in the upper position, the source of suction can be in fluid communication with the vent opening of the valve assembly such that the source of suction is open to room air.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts a cross-sectional side view of an illustrative suction valve assembly in a first position;

FIG. 3B depicts a cross-sectional side view of the illustrative suction valve assembly of FIG. 3A in a second position;

FIG. 4 depicts a cross-sectional side view of an illustrative suction valve assembly;

FIG. 5A depicts a perspective view of an illustrative suction valve assembly;

FIG. 5B depicts a cross-sectional side view of the illustrative suction valve assembly of FIG. 5A in a first position;

FIG. 5C depicts a cross-sectional side view of the illustrative suction valve assembly of FIGS. 5A and 5B in a second position;

FIG. 6A depicts the footprint of overlapping shafts for an illustrative suction valve assembly;

FIG. 6B depicts a partial cross-sectional side view of the illustrative suction valve assembly of FIG. 6A; and

FIG. 7 depicts a perspective view of an illustrative suction valve assembly.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

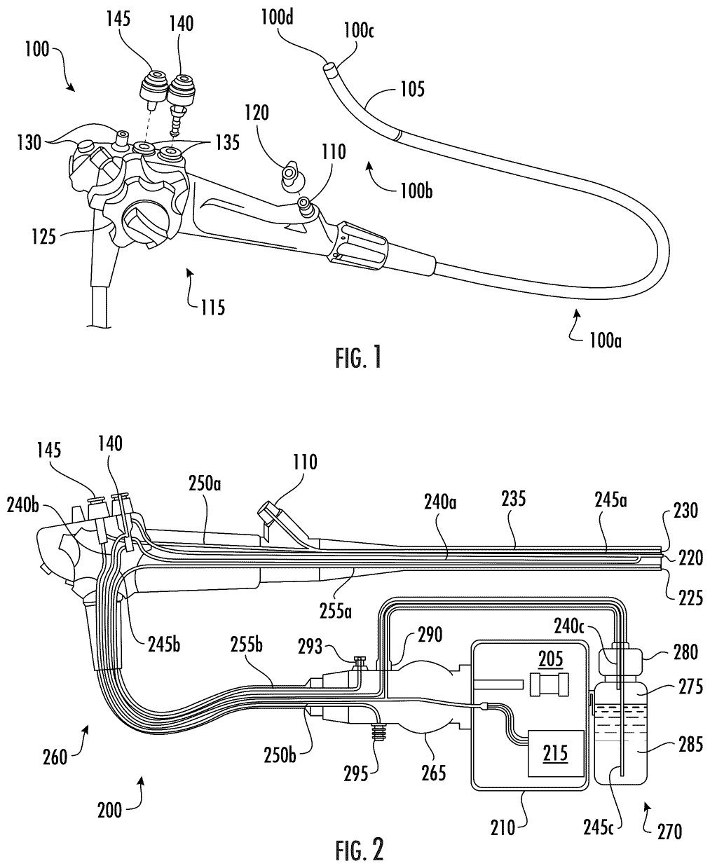

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilicus 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position)

In some cases, valve stems of suction valves 145 may be configured to have a close fit with a valve well configured to receive the valve stem in the endoscope 100. In such suction valves 145, when the valve stem is in a first position the close fit blocks a flow path or increases a resistance to flow between the working channel 235 and the suction pump and reduces a resistance to flow between atmosphere and the suction pump. Similarly, when the valve stem is in a second position, the close fit blocks a flow path or increases a resistance to flow between the atmosphere and the suction pump and reduces a resistance to flow between the working channel 235 and the suction pump.

Suction valves 145 configured to block flow using close fits between the valve stem and valve well requires valves stems that are precisely manufactured. The precision required to produce suction valves with close fits requires expensive materials (e.g., metals, etc.), highly precise machinery, and is time consuming to achieve.

Additionally, suction valves 145 with close fit valve stems and valve wells are manufactured to have at least some clearance to allow the valve stem to adjust positions within the valve well. This clearance may result in leakage during use, which may lead to two issues noticeable by a physician. The first is when the suction valve 145 is in a position intended to block suction from the working channel 235, there is still some suction flow passing through the working channel 235 and the suction valve 145 to the suction pump. The smaller the clearance between the valve stem and the valve well, the less unwanted flow through the working channel 235 that occurs and the larger the clearance, the more unwanted flow through the working channel 235, however, clearance is needed to facilitate movement of the valve stem within the valve well. When flow is actively moving up the working channel 235 in such configurations of the suction valve 235, users may perceive the suction as “poor insufflation” due to the suction of the suction pump pulling volume from a body lumen in which the user is working, even when the suction valve 145 is in a position intended to block a suction flow from the working channel 235. Second, when a valve stem of the suction valve 145 is in a position within a valve well to facilitate a suction flow between the working channel 235 and the suction pump through the suction valve 145, the flow from atmosphere to the suction pump may not be completely blocked. Any such leaking from atmosphere may reduce a pressure differential between suction valve and the distal end of the working channel 235, which leads to a reduced suction force or negative pressure, reduced flow rates, and aerated flow through the fluid path to the suction pump.

Suction valves 145 configured to operate with close-fit valve stems and valve wells may work well enough when intended for re-use in multiple procedures, as a price point for such suction valves can be high enough to justify manufacturing the suction valves 145 from materials and with the necessary precision that can achieve and maintain desired tolerances over the life of the reusable suction valves 145. However, a price point of a single use suction valve may not allow for use of the necessary materials, tools, and/or precise manufacturing required to achieve and/or maintain tolerances over the life of single-use suction valves.

The suction valve configurations for endoscopes 100 and/or other suitable scopes discussed herein address the above-noted concerns with existing suction valves and are configured to mitigate and/or eliminate leakage along an unintended flow path through the suction valve 145. The figures depict illustrative suction valve members capable of addressing these concerns.

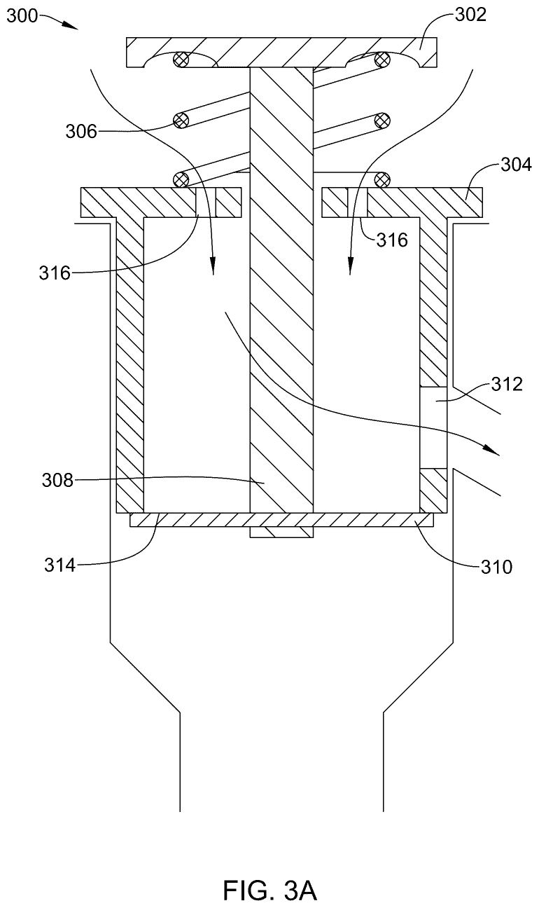

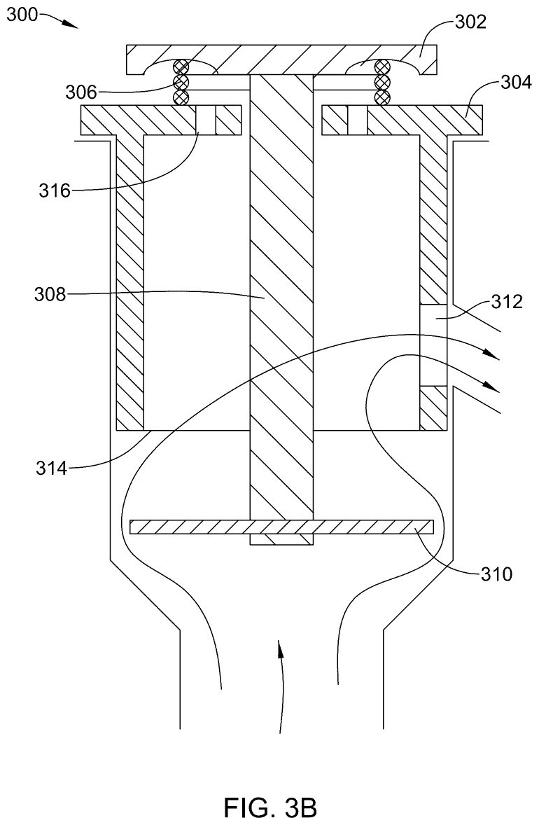

As shown in FIGS. 3A and 3B, a suction valve 300 includes a cap 302, a valve body 304, a spring member 306, a sliding shaft 308, and a bottom seal 310. The valve body 304 is inserted into the valve well as described above with respect to FIG. 2 and does not move relative to the valve well during the operation. An inlet opening 312 in the side of the valve body 304 is coupled to the suction channel as described, placing the valve body 304 in fluid communication with the source of suction.

The suction valve moves between a closed position, illustrated in FIG. 3A, and an open position, illustrated in FIG. 3B. In the closed position, the action of the spring member 306 biases the cap 302, sliding shaft 308, and bottom seal 310 upwards. The bottom seal 310 is seated against the bottom opening 314 of the valve body 304, blocking off the working channel of the valve well. Instead, the suction in fluid communication with the valve body 304 draws in room air through the vent openings 316 at the top of the valve body 304.

A user pressing downward on the cap 302 moves the valve 300 into an open position as shown in FIG. 3B. As the bottom seal 310 is no longer seated against the bottom opening 314 of the valve body 304, suction now extends through the valve body 304 and into the working channel as described above. The position of the cap 302 against the top surface of the valve body 304 also seals the vent openings 316 against suction while the valve 300 is in the open position. When the user releases the cap 302, the spring member 306 returns the valve 300 to the closed position.

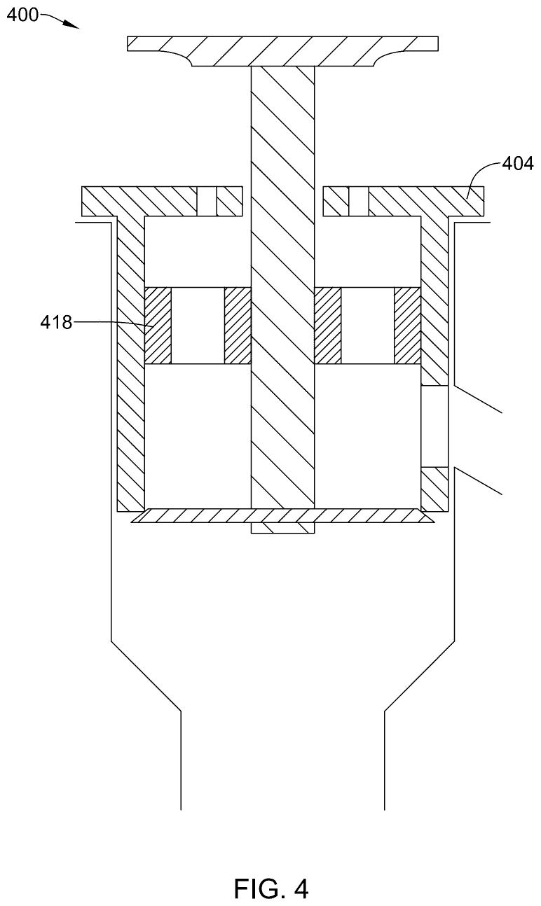

FIG. 4 shows a variation of the suction valve 400 in which the valve body 404 is made of a deformable material, such as a rubber, silicone, or TPE, with a significantly lower durometer and higher resilience than standard rigid materials. An insert 418, made of more rigid material, is located in the center of the valve body 404 to provide the necessary structure for the functioning of the valve. The flexible nature of the valve body allows it to fit tightly within the valve location 135 of the endoscope handle 1115.

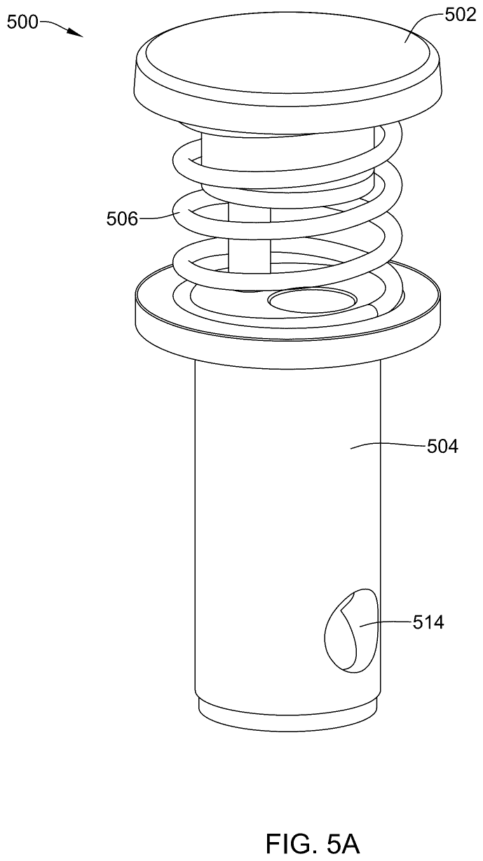

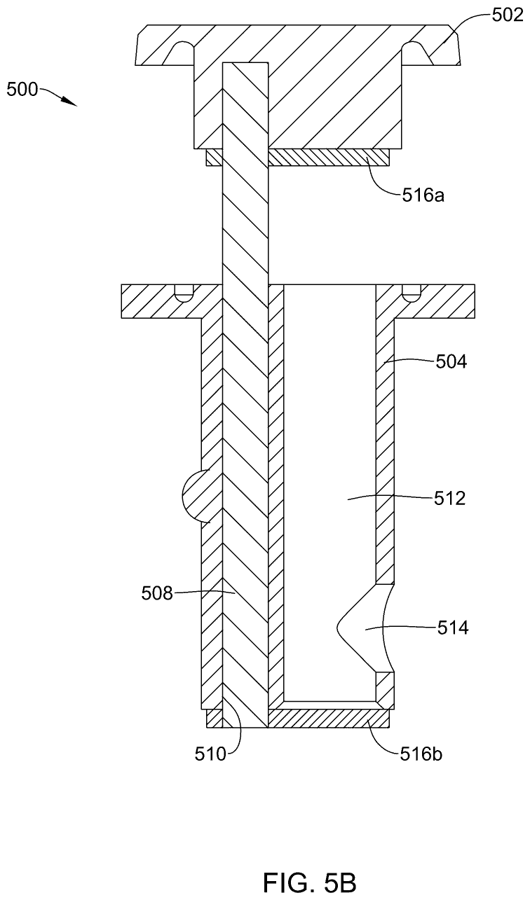

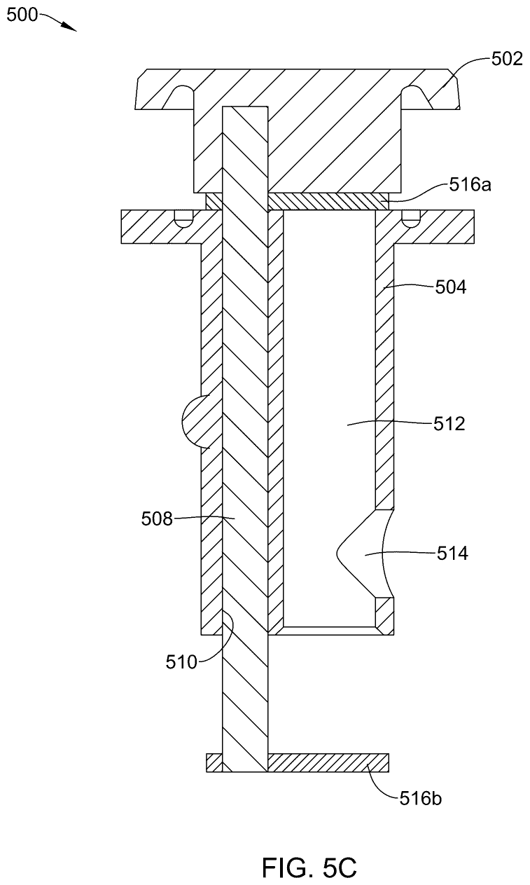

FIGS. 5A-5C show a suction valve 500 with a cap 502, a spring member 506, and a solid valve body 504. A sliding shaft 508, is disposed in a shaft channel 510. A vertical suction channel 512 opens at the top and bottom of the valve body 504 and communicates with an opening 514 that opens into the suction source as described above. The valve body 504 is stationary in the valve location, while the cap 502, seals 516a and 516b, and sliding shaft 508 translate between an upper closed position, where the suction channel is open to air, and a lower open position, where the suction is applied to the working channel. A flat seal 516a, located at the top of the vertical suction channel 512, obstructs air flow when the valve opens. A flat seal 516b, located at the bottom of the vertical suction channel 512, obstructs air flow when the valve closes.

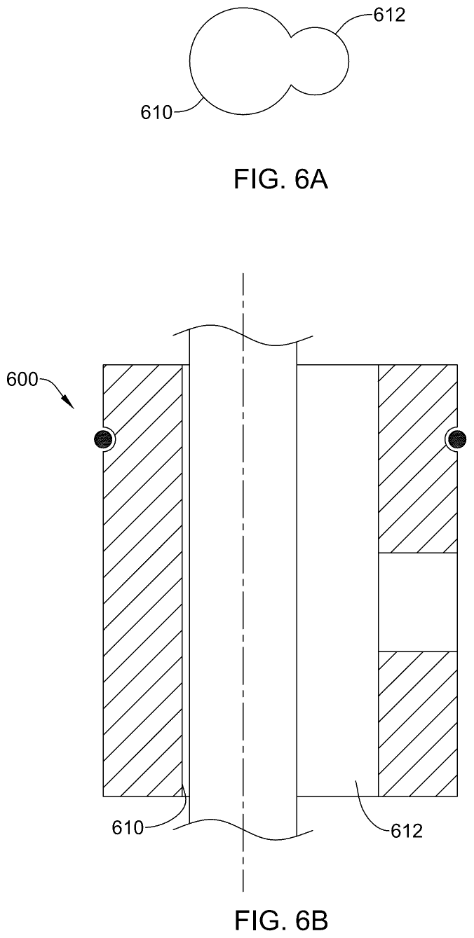

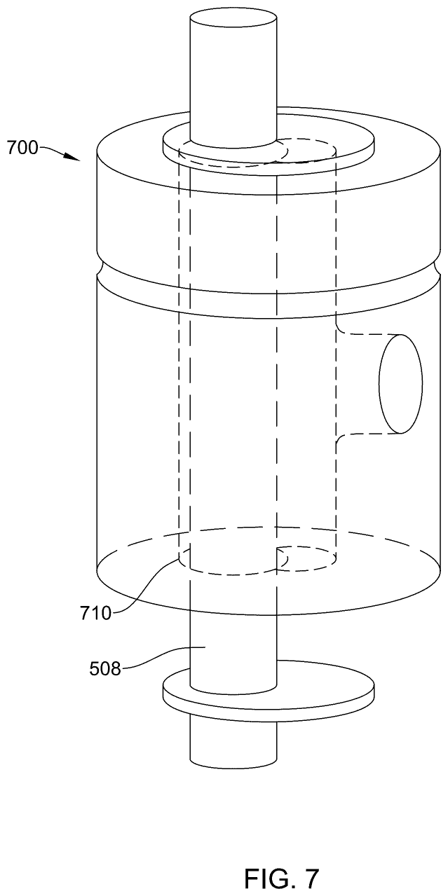

FIGS. 6A-B and 7 show valves 600 and 700 that are generally similar to the valve 500 but show variations in the vertical channels. In suction valve 600, the shaft channel 610 overlaps in footprint with the suction channel 612, such that the channels are open to each other and may be considered a single channel for purposes of fluid communication. Suction valve 700 has a single channel 710 that houses the sliding shaft 508 as well as acting as the channel for suction.

The valve member may be manufactured of relatively inexpensive materials suitable for disposal after a single use. The cap, neck, and shaft may be contiguously manufactured from a single piece of polyethylene plastic, each component may be manufactured from a separate piece of plastic and then attached together. Each of the components of the valve member may, in some implementations, be made of the same or similar materials, with the thickness and dimensions of each piece chosen to provide the necessary resilience and hardness.

In some implementations, the valve shaft may be injection-molded or extruded with one or more of the openings and/or slits not yet present, and then tooling, cutting, or another appropriate technique may be used to remove portions of the shaft in order to form the openings and/or slits. In some implementations, ribs, flanges, wedges, and other smaller features may be cut from the same original piece of material as the surrounding components or may be formed separately (of the same or different material) and then attached as shown.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A suction valve assembly for a medical device, comprising:
a cylindrical valve body having a top, a bottom, and a side surface, the valve body having a vent opening in the top, a working opening in the bottom, and a suction source opening in the side surface;
a valve cap;
a sliding shaft attached to the valve cap, the sliding shaft and valve cap moving vertically relative to the valve body between an upper position and a lower position; and
a lower seal attached to the sliding shaft, the seal seating against and obstructing the working opening when the sliding shaft is in the upper position, the lower seal below and not obstructing the working opening when the sliding shaft is in the lower position.

2. The suction valve assembly of claim 1, further comprising:
an upper seal moving with the sliding shaft and valve cap, the upper seal seating against and obstructing the vent opening when the sliding shaft is in the lower position, the upper seal above and not obstructing the vent opening when the sliding shaft is in the upper position.

3. The suction valve assembly of claim 2, wherein the upper seal is attached to the valve cap.

4. The suction valve assembly of claim 2, wherein the upper seal is attached to the sliding shaft.

5. The suction valve assembly of claim 1, the valve body further having a vertical air channel extending between the vent opening and the working opening and a side channel extending between the vertical air channel and the suction source opening.

6. The suction valve assembly of claim 5, the valve body further having a sliding shaft channel housing the sliding shaft.

7. The suction valve assembly of claim 6, wherein the sliding shaft channel is separated from the vertical air channel such that the sliding shaft channel is not in fluid communication with the vent opening, working opening, or suction source opening.

8. The suction valve assembly of claim 6, wherein the sliding shaft channel overlaps the vertical air channel such that the sliding shaft channel is in fluid communication with the vent opening, working opening, or suction source opening.

9. The suction valve assembly of claim 1, further comprising:
a spring member positioned between the valve body and the valve cap, the spring member biased to push the valve cap and sliding shaft upward into the upper position.

10. The suction valve assembly of claim 1, further comprising:
a rigid insert positioned within the valve body, the rigid insert having a slide shaft opening for movement of the slide shaft therethrough and an air opening for movement of air therethrough when the slide shaft is in the upper position;
wherein the valve body is made of a flexible material having a lower durometer than the rigid insert.

11. The suction valve assembly of claim 1, further comprising a first outer seal positioned on the side surface of the valve body above the suction source opening and a second outer seal positioned on the side surface of the valve body below the suction source opening.

12. The suction valve assembly of claim 1, wherein at least one of the first and second outer seals is a radial o-ring.

13. An endoscopic surgical device, comprising:
an endoscopic probe having a working channel;
the suction valve assembly, comprising:
a cylindrical valve body having a top, a bottom, and a side surface, the valve body having a vent opening in the top, a working opening in the bottom in fluid communication with the working channel of the endoscopic probe, and a suction source opening in the side surface,
a valve cap,
a sliding shaft attached to the valve cap, the sliding shaft and valve cap moving vertically relative to the valve body between an upper position and a lower position, and
a lower seal attached to the sliding shaft, the seal seating against and obstructing the working opening when the sliding shaft is in the upper position, the lower seal below and not obstructing the working opening when the sliding shaft is in the lower position; and
a source of suction in fluid communication with the suction source opening of the suction valve assembly, such that, when the sliding shaft of the valve is in the lower position, it provides suction to the working channel of the endoscopic probe from the suction source opening, through the valve body, and into the working channel opening.

14. The endoscopic surgical device of claim 13, further comprising an endoscopic handle having a suction valve location with an inner wall, the side surface of the body valve of the suction valve assembly pressing tightly against the inner wall of the suction valve location of the endoscopic handle.

15. The endoscopic surgical device of claim 13, wherein when the sliding shaft of the valve is in the upper position, the source of suction is in fluid communication with the vent opening of the valve assembly such that the source of suction is open to room air.

16. The endoscopic surgical device of claim 13, the suction valve assembly further comprising:
an upper seal moving with the sliding shaft and valve cap, the upper seal seating against and obstructing the vent opening when the sliding shaft is in the lower position, the upper seal above and not obstructing the vent opening when the sliding shaft is in the upper position.

17. The endoscopic surgical device of claim 13, the valve body of the suction valve assembly further having a vertical air channel extending between the vent opening and the working opening and a side channel extending between the vertical air channel and the suction source opening.

18. The endoscopic surgical device of claim 17, the valve body of the suction valve assembly further having a sliding shaft channel housing the sliding shaft.

19. The endoscopic surgical device of claim 18, wherein the sliding shaft channel is separated from the vertical air channel such that the sliding shaft channel is not in fluid communication with the vent opening, working opening, or suction source opening.

20. The suction valve assembly of claim 19, wherein the sliding shaft channel overlaps the vertical air channel such that the sliding shaft channel is in fluid communication with the vent opening, working opening, or suction source opening.
